(19) United States
(12) Patent Application Publication
(10) Pub. No.: US 2008/0200699 A1
(43) Pub. Date: Aug. 21, 2008

(54) Method for Preparing Docetaxel

(76) Inventors: Antoine Paul Gatson Leze, La Milesse (FR); Regis Perion, Le Mans (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/816,915

(22) PCT Filed: Feb. 21, 2006
(86) PCT No.: PCT/FR06/00386

§ 371 Date: Apr. 8, 2008

(30) Foreign Application Priority Data
Feb. 24, 2005 (FR) 0501882

(51) Int. Cl. C07D 305/14

(52) U.S. Cl. 549/510

(57) ABSTRACT

The invention concerns a method for preparing docetaxel from paclitaxel, including the following steps: a) deacylating paclitaxel, b) protecting the free hydroxy functions, in 7-, 10- and 2′-position respectively, c) debenzoylating the amine in 3′-position, into a primary amine derived for 10-deacetylbaccatine whereof the hydroxy functions in 7-, 10- and 2′-position are protected, d) functionalizing the amine with a t-butoxycarbonyl In radical to obtain a docetaxel derivative of general formula (I), wherein: X represents protecting radicals or hydrogen atoms, then, if required, e) releasing the initially protected hydroxy functions to obtain docetaxel.

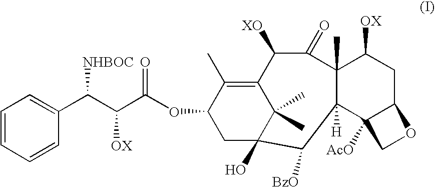

The present invention relates to a process for preparing docetaxel from paclitaxel.

Processes for preparing docetaxel have already been widely described. However, all the processes do not always start from a readily accessible starting material, or are not easily industrializable.

Paclitaxel is now widely available on the market, which may, besides its therapeutic value, make it an advantageous starting material that is readily accessible and of constant and reproducible purity.

International patent application WO 96/23780 describes a process that consists in preparing the primary amine of paclitaxel and its conversion into paclitaxel or other taxane derivatives, especially docetaxel. However, the tests are never performed for the purpose of preparing a primary amine of 10-deacetyl paclitaxel protected on the hydroxyl functions in positions -7,-10 and -2′, and the conversion of such an amine into docetaxel protected in positions -7,-10 and -2′ or thereafter into docetaxel is not described. The implementation of the described process is difficult to transpose to the industrial scale.

It has now been found, and this is the subject of the present invention, that docetaxel can be prepared from paclitaxel by performing the following steps:
a) deacetylation of paclitaxel of formula:

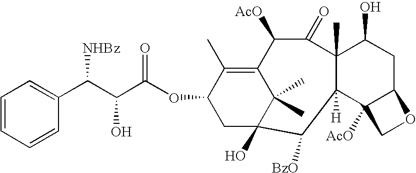

b) protection of the free hydroxyl functions, in positions -7,-10 and -2′, respectively,
c) debenzoylation of the amine in position -3, to a primary amine derived from 10-deacetylbaccatin whose hydroxyl functions in positions -7, -10 and -2′ are protected,
d) functionalization of the amine with a t-butoxycarbonyl radical to obtain a docetaxel derivative of general formula:

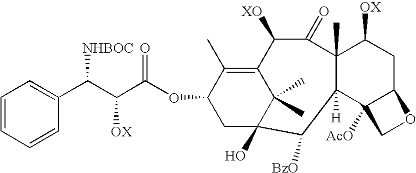

e) in which X represents protecting radicals or hydrogen atoms, e) and then, where appropriate, release of the initially protected hydroxyl functions to obtain docetaxel of formula:

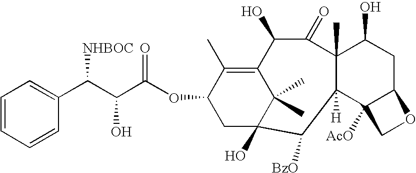

Advantageously, the deacetylation in step a) is performed via the action of a peroxide in basic medium.

The peroxide used is chosen from the peroxides usually used industrially and that do not impair the rest of the molecule. It is especially chosen from hydrogen peroxide, tert-butyl peroxide, meta-chloroperbenzoic acid and a monoperoxyphthalic acid salt. Hydrogen peroxide is preferably used.

The base is preferably chosen from alkali metal or alkaline-earth metal carbonates or hydroxides. More particularly, and in a nonlimiting manner, the base is chosen from calcium carbonate, sodium bicarbonate, sodium carbonate, potassium carbonate, barium carbonate, cesium carbonate, sodium hydroxide, potassium hydroxide, lithium hydroxide, barium hydroxide, calcium hydroxide and magnesium hydroxide.

The reaction is performed in water or in an organic solvent, for instance an ether (for example tetrahydrofuran), an alcohol (for example methanol or ethanol), a ketone (for example acetone) or in a chlorinated solvent (for example dichloro-methane, dichloroethane or chloroform) at a temperature of between 0 and 50° C. and preferably at a temperature of between 20 and 25° C. The process is advantageously performed under a nitrogen atmosphere.

Protection of the hydroxyl radicals at -7,-10 and -2′, respectively, with protecting radicals X is performed according to any known method used in taxane chemistry and for which the installation and removal of the protecting radicals does not impair the rest of the molecule. The hydroxyl-protecting radicals may be chosen especially from silyl radicals, for instance trialkylsilyl, dialkylarylsilyl, alkyldiarylsilyl or triarylsilyl groups in which the alkyl radicals contain 1 to 4 C in a straight or branched chain and aryl is preferably phenyl, for example triethylsilyl or trimethylsilyl radicals, or chlorinated radicals, for instance trichloroalkyloxycarbonyl radicals, for example trichloroethoxycarbonyl, especially 2,2,2-trichloroethoxy-carbonyl or trichloromethylpropoxycarbonyl, especially 2-(2-trichloromethyl-propoxy)carbonyl. A halide (for example the chloride) of a trialkylsilane, dialkylarylsilane, alkyldiarylsilane or triarylsilane is especially reacted in the presence of a nitrogenous base, for instance triethylamine, dimethylaminopyridine, pyridine or imidazole, in an organic solvent, for instance an amide (for example dimethylformamide). The process is preferably performed under a nitrogen atmosphere.

Step c) of debenzoylation of the amine function to a primary amine may be performed according to the method described by Kingston, Tetrahedron Letters, 40, 189-192 (1999), which is incorporated herein by way of reference, especially using an alkali metal or alkaline-earth metal alkoxide, for instance magnesium methoxide or magnesium ethoxide. Magnesium methoxide is preferably used.

According to another embodiment of the invention, step c) of debenzoylation of the amine function to a primary amine may also be performed via the action of a reductive amount of Schwartz reagent: CP₂ZrHCl (zirconocene hydrochloride) for which Cp means cyclopentadienyl, followed by hydrolysis of the imine obtained. The reaction is advantageously performed in a solvent such as an ether (for example tetrahydrofuran) at a temperature of between 15 and 25° C., preferably under a nitrogen atmosphere.

Hydrolysis of the imine is performed in a medium of aqueous mineral acid dissolved in a protic solvent chosen from alcohols and ethers. The mineral acid may be chosen from hydrochloric acid and hydrobromic acid. The protic solvent may be advantageously chosen from an alcohol (for example ethanol, methanol, etc.) and an ether (for example tetrahydrofuran or dioxane). It is understood that when the process is performed on a paclitaxel derivative whose hydroxyl functions at -7,-10 and -2′ are protected with silyl radicals, said radicals are simultaneously removed in the hydrolysis reaction.

Step d) of functionalization of the amine with a t-butoxycarbonyl radical is performed according to the known methods for the formation of carbamates from an amine function, which do not impair the rest of the molecule. It is especially possible to react tert-butoxycarboxylic acid anhydride ((CH₃)₃C—O—CO)₂O with the primary amine of general formula:

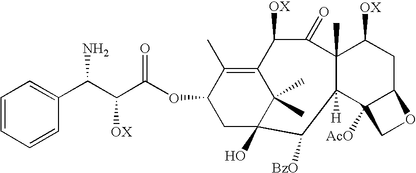

in which X represents protecting radicals as defined above or represents hydrogen atoms, working under the usual conditions that do not impair the rest of the molecule. The process is especially performed by addition of di-tert-butyl dicarbonate at a controlled pH of 7.5, by addition of sodium hydroxide, in an alcohol, for instance methanol, or in a water/ester (for example water/ethyl acetate) aqueous-organic medium, in the presence of a carbonate or a bicarbonate (for example sodium bicarbonate).

According to the invention, the paclitaxel used as starting material may be advantageously prepared from 10-deacetylbaccatin according to a “one-pot” reaction involving the three successive steps of protecting the hydroxyl radical in position -7, of acetylation of the hydroxyl radical in position-10, and of crystallization of the baccatin III derivative obtained, followed by condensation of (4S,5R)-3-N-benzoyl-2RS-methoxy-4-phenyl-1,3-oxazolidine-5-carboxylic acid of formula:

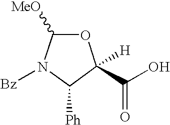

by esterification in position-13 of the 10-acetyl derivative obtained of general formula:

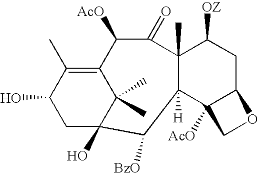

in which Z is a protecting radical, to obtain a derivative of general formula;

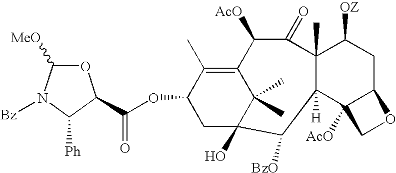

in which Z is a protecting radical, followed by opening the oxazolidine of the cyclic side chain and simultaneous release of the hydroxyl radical in position-7 from its protecting radical and optional purification of the paclitaxel thus obtained.

The protecting radical Z is chosen from protecting radicals usually used in taxane chemistry, whose installation and removal do not impair the rest of the molecule. The hydroxyl-protecting radicals are especially as mentioned above for X.

The operating conditions for the preparation of the paclitaxel used as starting material, from 10-deacetylbaccatin, are described in international patent application WO 94/07878.

The example that follows is given as a nonlimiting illustration of the invention.

### EXAMPLE

### Preparation of 1 0-deacetylpaclitaxel

Paclitaxel (6.9 g, 8.08 mmol) is dissolved at 20° C. in 138 ml of tetrahydrofuran in a 500 ml three-necked flask, under a nitrogen atmosphere. 30% aqueous hydrogen peroxide solution (solution cooled to 5° C.) (138 ml, 1.22 mol, >150 eq.) and then sodium hydrogen carbonate (13.2 g, 0.16 mmol, 19 eq.) are added to the reaction mixture. After stirring for 24 hours, the reaction mixture is diluted with dichloromethane (500 ml). The organic phase is washed with water (500 ml). The aqueous phase is extracted once with 250 ml of dichloromethane. The organic phase is then dried over MgSO₄, filtered and then evaporated under reduced pressure. The residue taken up in dichloromethane is purified by chromatography (96/4 CH₂Cl₂/MeOH) to give 6.5 g (99%) of the expected product.

TLC: Rf=0.4 (93/7 CH₂Cl₂/MeOH).

### Preparation of 2′,7,10-tris(triethylsilyl)paclitaxel

Triethylsilane chloride (206 μl, 1.23 mmol, 10 eq.) and imidazole (100 mg, 1.47 mmol, 12 eq.) are added to 10-deacetylpaclitaxel (100 mg, 0.123 mmol) dissolved in 1 ml of anhydrous dimethylformamide in a 10 ml three-necked flask, under a nitrogen atmosphere. The reaction mixture is heated at 57° C. for 7 hours. After cooling to room temperature, the mixture is diluted with 20 ml of ethyl acetate. The organic phase is washed with water (20 ml), dried over MgSO₄, filtered and then evaporated under reduced pressure. The crude product is purified by chromatography (90/10 and then 80/20 cyclohexane/ethyl acetate). The combined fractions are concentrated under reduced pressure (106 mg) to give 106 mg (75%) of the expected product.

35 TLC: Rf=0.3 (80/20 cyclohexane/ethyl acetate).

Mass spectrum: m/z=1176.53 [M+Na]⁺

¹H NMR (CDCl₃, 400 MHz) δ (ppm): 0.39-0.85 (m, 27H, 9×CH₃); 0.95-1.02 (m, J 8H, 9×CH₂); 1.18 and 1.22 (2s, 6H, CH₃-16 and CH₃-17); 1.68 (s, 3H, CH₃ 19); 1.69 (s, 1H, OH-1); 1.86 (s, 3H, CH₃-18); 1.92 (m, 1H, H6-B); 2.13-2.39 (m, 2H, CH₂-14); 2.53 (s, 3H, AcO-4); 2.54 (m, 1H, H6-A)°; 3.86 (d, 1H, J=7.0 Hz, H3); 4.20-4.32 (2d, 2H, J=8.4 Hz, H20); 4.40 (m, 1H, H7); 4.69 (d, 1H, J=2.0 Hz, H2′); 4.93 (d, 1H, J=8.1 Hz, H5); 5.16 (s, 1H, H10); 5.66-5.70 (m, 2H, H2+H3′); 6.22 (t, 1H, H13); 7.14 (d, 1H, J=8.7 Hz, NH); 7.32-7.43 (m, 7H, m-PhCONH, Ph); 7.45-7.53 (m, 3H, m-PhCOO and p-PhCONH); 7.60 (t, 1H, J=7.4 Hz, p-PhCOO); 7.77 (d, 2H, J=7.1 Hz, o-PhCONH); 8.12 (d, 2H, J=7.2 Hz, o-PhCOO).

### Preparation of N-debenzoyl-10-deacetylpaclitaxel

Schwartz reagent (Cp₂ZrHCl; 41.5 mg, 0.161 mmol, 3 eq.) is added to a solution of 2′,7,10-tris(triethylsilyl)paclitaxel (62 mg, 0.0537 mmol) in 620 μl of anhydrous tetrahydrofuran in a 10 ml round-bottomed flask, under a nitrogen atmosphere. After stirring for one hour at a temperature of 24° C., the reaction mixture is homogeneous. After stirring for a further 30 minutes, the mixture is poured into 15 ml of cold (4° C.) cyclohexane. A white veil forms. Cold cyclohexane (10 ml) is added. The mixture is left at 4° C. for 1 hour 30 minutes and then filtered. The filtrate is concentrated to dryness and then dissolved in 3.1 ml of methanol. Aqueous 1N hydrochloric acid solution (268 μl, 0.268 mmol, 5 eq.) is added. After stirring for 3 hours, a further 2 equivalents of 1N hydrochloric acid are added (107 μl, 0.107 mmol, 2 eq.). The mixture is diluted with cyclohexane (20 ml). The organic phase is washed with water (20 ml). Addition of 5 ml of ethyl acetate breaks the emulsion formed during the washing. The organic phase is washed with 5 ml of aqueous sodium hydrogen carbonate solution (5%). The aqueous phase is extracted three times with 15 ml of dichloromethane. The organic phases are combined, dried over MgSO₄, filtered and then evaporated under reduced pressure. The crude product is purified by chromatography (93/7 CH₂Cl₂/MeOH). The combined fractions are concentrated under reduced pressure to give 27 mg (71%) of the expected product.

TLC: Rf=0.3 (93/7 CH₂Cl₂/MeOH).

Mass spectrum: m/z=708.26 [M+H]⁺
m/z=730.23 [M+Na]⁺

### Preparation of Docetaxel:

The deprotected compound (25 mg, 0.035 mmol) is dissolved in 3.5 ml of ethyl acetate and 3.5 ml of saturated aqueous sodium hydrogen carbonate solution in a 10 ml round-bottomed flask. Di-tert-butyl dicarbonate (15.4 mg, 0.071 mmol, 2 eq.) is added. After stirring for 5 hours 30 minutes, the reaction mixture is diluted with 7 ml of ethyl acetate. The organic phase is washed twice with water (7 ml). The organic phases are combined, dried over MgSO₄, filtered and then evaporated under reduced pressure. The crude product is purified by chromatography (95/5 CH₂Cl₂/MeoH). The combined fractions are concentrated under reduced pressure to give 22 mg (78%) of the expected product.

TLC: Rf=0.2 (93/7 CH₂Cl₂/MeOH).

Mass spectrum: m/z=830.13 [M+Na]⁺

¹H NMR (CDCl₃, 400 MHz) δ (ppm): 1.13 and 1.24 (2s, 6H, CH₃-16 and CH₃-17); 1.34 (s, 9H, tBu); 1.74 (s, 1H, OH-1); 1.75 (s, 3H, CH₃-19); 1.85 (s, 3H, CH₃-18); 1.81-1.87 (m, 1H, H6-B); 2.27 (d, 2H, J=8.4 Hz, CH₂-14); 2.37 (s, 3H, AcO-4); 2.54-2.62 (m, 1H, H6-A)°; 3.42 (broad s, 1H, OH-2′); 3.91 (d, 1H, J=7.0 Hz, H3); 4.18-4.32 (m, 4H, H20, H7 and OH-10); 4.62 (s, 1H, H2′); 4.94 (d, 1H, J=7.9 Hz, H5); 5.21 (s, 1H, H10); 5.25 (d, 1H, J=8.5 Hz, H3′); 5.45 (d, 1H, J=9.4 Hz, NH); 5.67 (d, 1H, J=7.0 Hz, H2); 6.21 (t, 1H, J=8.6 Hz, H13); 7.30-7.42 (m, 5H, Ph); 7.48-7.52 (m, 2H, J=7.5Hz, m-PhCOO); 7.61 (t, 1H, J=7.4 Hz, p-PhCOO); 8.11 (d, 2H, J=7.4 Hz, o-PhCOO).

1. A process for preparing docetaxel from paclitaxel, characterized in that the following steps are performed:
a) deacetylation of paclitaxel of formula: 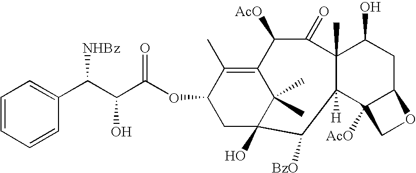
b) protection of the free hydroxyl functions, in positions-7, -10 and -2′, respectively,
c) debenzoylation of the amine in position-3′ to a primary amine derived from 10-deacetylbaccatin whose hydroxyl functions in positions-7, -10 and -2′ are protected,
d) functionalization of the amine with a t-butoxycarbonyl radical to obtain a docetaxel derivative of general formula: 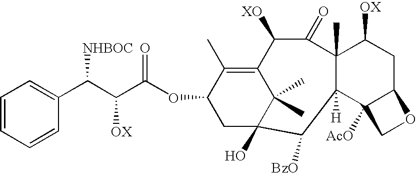
in which X represents protecting radicals or hydrogen atoms, and then, where appropriate,
e) release of the initially protected hydroxyl functions to obtain docetaxel of formula:

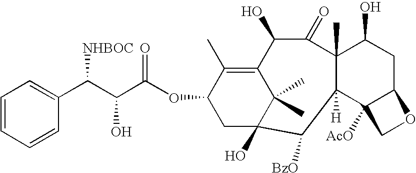

2. The process as claimed in claim 1, characterized in that the paclitaxel used as starting material is prepared from 10-deacetylbaccatin according to a “one-pot” reaction involving the three successive steps of protecting the hydroxyl radical in position-7, of acetylation of the hydroxyl radical in position -10, and of crystallization of the baccatin III derivative obtained, followed by condensation of (4S,5R)-3-N-benzoyl-2RS-methoxy-4-phenyl-1,3-oxazolidine-5-carboxylic acid of formula: 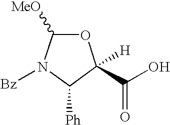
by esterification in position-13 of the 10-acetyl derivative obtained of general formula: 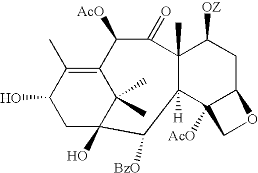
in which Z is a protecting radical, to obtain a derivative of general formula: 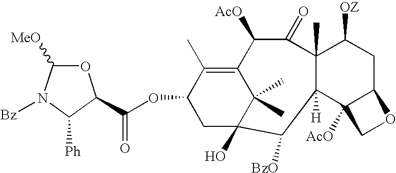
in which Z is a protecting radical, followed by opening the oxazolidine of the cyclic side chain and simultaneous release of the hydroxyl radical in position-7 from its protecting radical and optional purification of the paclitaxel thus obtained.

3. The process as claimed in claim 1, characterized in that the deacetylation is performed via the action of a peroxide in basic medium.

4. The process as claimed in claim 3, characterized in that the peroxide is chosen from hydrogen peroxide, tert-butyl peroxide, meta-chloroperbenzoic acid and a monoperoxyphthalic acid salt.

5. The process as claimed in either of claim 3, characterized in that the base is chosen from alkali metal or alkaline-earth metal carbonates or hydroxides.

6. The process as claimed in claim 5, characterized in that the base is chosen from calcium carbonate, sodium bicarbonate, sodium carbonate, potassium carbonate, barium carbonate, cesium carbonate, sodium hydroxide, potassium hydroxide, lithium hydroxide, barium hydroxide, calcium hydroxide and magnesium hydroxide.

7. The process as claimed in claim 1, characterized in that the hydroxyl-protecting radicals are chosen from silyl and trichloroalkyloxycarbonyl radicals.

8. The process as claimed in claim 7, characterized in that the silyl radicals are chosen from trialkylsilyl, dialkylarylsilyl, alkyldiarylsilyl and triarylsilyl groups in which the alkyl radicals contain 1 to 4 C in a straight or branched chain and aryl represents phenyl, preferably triethylsilyl or trimethylsilyl, and in that the trichloroalkyloxycarbonyl radicals are chosen from trichloroethoxycarbonyl and trichloromethylpropoxycarbonyl groups.

9. The process as claimed in claim 1, characterized in that step c) of debenzoylation of the amine function, to a primary amine, is performed via the action of an alkali metal or alkaline-earth metal alkoxide.

10. The process as claimed in claim 9, characterized in that the alkoxide is chosen from magnesium methoxide and magnesium ethoxide.

11. The process as claimed in claim 1, characterized in that step c) of debenzoylation of the amine function, to a primary amine, is performed via the action of a reductive amount of Schwartz reagent: Cp₂ZrHCl for which Cp means cyclopentadienyl, followed by hydrolysis of the imine obtained.

12. The process as claimed in claim 11, characterized in that the hydrolysis of the imine is performed in a medium of aqueous mineral acid dissolved in a protic solvent chosen from alcohols and ethers.

13. The process as claimed in claim 12, characterized in that the mineral acid is chosen from hydrochloric acid and hydrobromic acid, and the protic solvent is chosen from ethanol, methanol, tetrahydrofuran and dioxane.

14. The process as claimed in claim 1, characterized in that step d) of functionalization of the amine with a t-butoxycarbonyl radical is performed according to the known methods for formation of carbamates from an amine function, which do not impair the rest of the molecule.

15. The process as claimed in claim 14, characterized in that tert-butoxycarboxylic acid anhydride ((CH₃)₃C—O—CO)₂O is reacted with the primary amine of general formula: 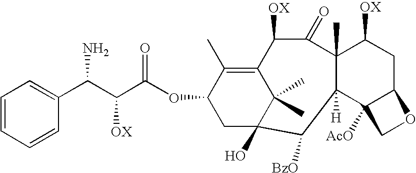
in which X represents protecting radicals or hydrogen atoms.

16. The process as claimed in claim 15, characterized in that the reaction is performed at a controlled pH of 7.5, dissolved in an alcohol.

17. The process as claimed in claim 15, characterized in that the reaction is performed in water/ester (for example water/ethyl acetate) aqueous-organic medium, in the presence of a carbonate or a bicarbonate.

18. The process as claimed in claim 1, characterized in that paclitaxel is deacetylated, under a nitrogen atmosphere, with aqueous 30% hydrogen peroxide solution in the presence of sodium hydrogen carbonate, at 20° C. in tetrahydrofuran, and positions-7, -10 and -2′ of 10-deacetylpaclitaxel are then protected with triethylsilyl radicals via the action of triethylsilane chloride in dimethylformamide in the presence of imidazole, the 2′,7,10-tris(triethylsilyl)paclitaxel is then debenzoylated via the action of Schwartz reagent (Cp₂ZrHCl) in anhydrous tetrahydrofuran, under a nitrogen atmosphere, at a temperature of 24° C., and the imine obtained is treated with IN hydrochloric acid, and docetaxel is then prepared from the deprotected compound obtained, by treatment with di-tert-butyl dicarbonate in the presence of saturated aqueous sodium hydrogen carbonate solution, in ethyl acetate.
